(19) United States
(12) Patent Application Publication
(10) Pub. No.: US 2012/0029270 A1
(43) Pub. Date: Feb. 2, 2012

(54) CERVICAL CONCEPTION CONDOM

(76) Inventor: CHRISTINE STUART, Lubbock, TX (US)

(21) Appl. No.: 12/848,154

(22) Filed: Jul. 31, 2010

(51) Int. Cl. A61B 17/425

(52) U.S. Cl. 600/33

(57) ABSTRACT

The present invention relates to a cervical condom for aiding in conception comprising: a dome shape flexible device, where said device is made of a flexible material; and a rim surrounding the outer perimeter of the device. The device is capable of containing a sperm sample for application upon a user's cervix. In one exemplary embodiment, the flexible material is silicone and the condom measures in a range of about two to five centimeters in length.

### BACKGROUND OF THE INVENTION

1. Field of Invention

The present invention relates to a cervical conception condom that is designed to retain semen in and around the cervix area to enhance the likelihood of conception.

2. Description of Related Art

Many women struggle with fertility and go to great lengths to achieve conception. Various factors are considered when trying to achieve conception and various techniques are used to ensure conception. Some women try forms of artificial insemination where sperm is injected into the cervical area to achieve conception. Many times during this process sperm specimen is forced into the uterus in hopes of fueling fertilization. This process sometimes strips the seminal fluids of its nutrients. The seminal fluids are stripped away from the sperm when the user's body temperature is inappropriate or she is in an unrelaxed condition. Also the insemination must take place when the woman is ovulating. However many of these techniques aren't as effective to suit the needs of a consumer and many women may struggle to conceive month after month.

The further drawbacks to the artificial insemination are the costs associated with artificial insemination. Many procedures are costly and many insurance companies do not provide coverage for such procedures. One technique used to increase the likelihood of conception is the placement of semen near the cervix to allow the specimen to enter the woman's uterus. The process of conception requires that the seminal specimen be placed in the right position and path and not forced into the woman's cervix or uterus. The forcing causes a woman's body to fight off and actually reject the semen that is being injected.

Consequently there is a need for an inexpensive device that could be used by a woman in conjunction with her mate to facilitate conception. Such a device should be easy to use, practical and easy to insert and place against a woman's cervix. The device should be able to hold the sample semen and allow the woman to apply the semen directly to the cervix in a relaxed manner and in her bedroom.

### SUMMARY OF THE INVENTION

The present invention relates to a cervical condom for aiding in conception comprising: a dome shape flexible device, where said device is made of a flexible material; and a rim surrounding the outer perimeter of the device. The device is capable of containing a sperm sample for application upon a user's cervix. In one exemplary embodiment, the flexible material is silicone and the condom measures in a range of about two to five centimeters in length.

### BRIEF DESCRIPTION OF DRAWINGS

FIG. 1 depicts the cervical conception condom according to the present invention.

### DETAIL DESCRIPTION

The present invention relates to a cervical conception condom that is used to enhance the likelihood of conception. The conception condom according to the present invention contains a sperm sample for insertion directly onto a woman's cervix. The cervical condom is a device that is made of a flexible material in the shape of a small dome. The conception condom is designed to retain the semen for placement in and near the cervical canal therefore increasing the likelihood of conception.

The present invention helps to maximize the likelihood of conception when a woman is ovulating or just prior to ovulation by placing the semen in the correct area near the cervical opening. The present invention increases the likelihood of the uterus receiving sperm while the cervical condom is held in place inside the warm internal vagina and cervical area of the female. The cervical condom is made of a flexible material, which is collapsible but has adequate tensile strength for the containment and insertion of the semen onto the woman's cervix. The cervical conception condom also conforms to a particular woman's shape and size of her cervix in order to have appropriate fit for maximum application of sperm.

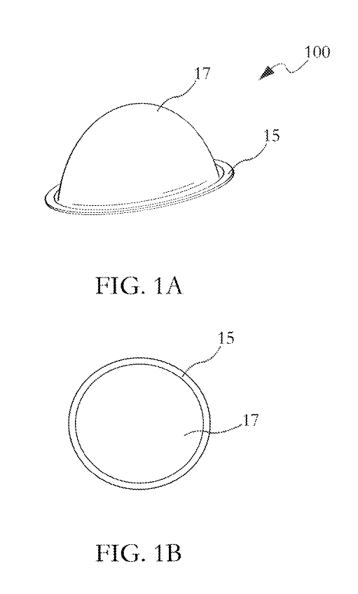

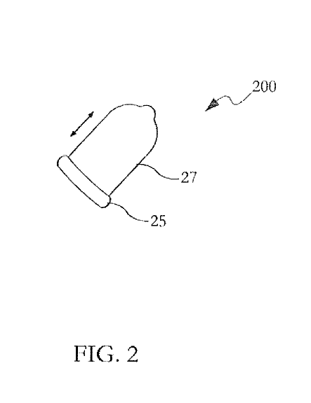

Referring to FIG. 1 a dome-shaped Conception Condom 100 is depicted in accordance with the present invention. The cervical conception condom is a dome-shaped device that is made from a flexible sheath like material and in one particular embodiment may be made of silicon that contains sperm for application over a woman's cervix. The use of silicon helps to lower risks of allergic reactions as oppose to using latex. The Cervical Condom 100 includes the Dome Body 15 and an Outer Rim 12. The Outer Rim 12 defines the perimeter of the Cervical Condom 100. The Outer Rim 12 unrolls to adjust the length of the Condom 100 for longer cervices. An opening is provided within the Dome 15 for the insertion of sperm at the time of use. The Cervical Condom 100 functions in a manner that is contrary to the traditional condom which is used to prevent the insertion of sperm into the cervical and uteral area of the female, in contrast the Cervical Condom 100 contains the sperm for direct application over the woman's cervix. In one particular embodiment according to the present invention, the Cervical Condom 100 may be two to five centimeters in length but however the actual sizes would vary to accommodate the different sizes of cervices on the women.

The use of the Cervical Condom 100 helps to increase the likelihood of conception, which is normally at or about 20 percent chance per female ovulation cycle. These odds may increase through the use of the Cervical Condom 100 according to the present invention. The application of the seminal fluid around the cervical area at the time of a woman's ovulation helps to increase the likelihood of conception due to the openness and receptiveness of the cervical tissue at this particular time. Use of this cervical condom by placing the semen directly onto the cervical area hastens the transmission of sperm through the cervical labyrinths and into the uterus. Use of the cervical condom according to present invention helps to eliminate some of the problems with the traditional artificial insemination methods that are costly and not as effective as the use of the cervical condom according to the present invention. Further the cervical condom may be used in the privacy of a woman's home with the assistance of their mate to place the cervical condom directly against the cervix. The cervical condom may be used at bedtime and therefore increase the likelihood or maximize the angle application on to the woman's cervix. Further the cervical condom can help minimize frustration associated with fruitless attempts to conceive and give a woman a great tool and advantage in enhancing the likelihood of conception.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

1. A cervical condom for aiding in conception comprising:
a. a dome shape flexible sheath-like device, where said device is made of a flexible material; and
b. a rim surrounding the outer perimeter of the device;

2. The cervical condom according to claim 1, where said device is capable of containing a sperm sample for application upon a user's cervix.

3. The cervical condom according to claim 1, where said flexible material is silicone.

4. The cervical condom according to claim 1, where said condom measures in a range of about two to five centimeters in length.

5. A method of applying a cervical condom comprising the steps of:
inserting a sperm specimen within a reservoir tip of a dome body of the cervical condom;
manually applying the cervical condom onto a user's cervix;
extending the overall length of the condom to securely place the condom onto the user's cervix; and
ensuring that the outer rim of the cervical condom is firmly placed over the circumference of the cervix.
